(19) United States
(12) Patent Application Publication
(10) Pub. No.: US 2007/0038020 A1
(43) Pub. Date: Feb. 15, 2007

(54) Endoscope apparatus

(76) Inventor: Der-Yang Tien, Taipei (TW)

Correspondence Address: MORLAND C FISCHER, 2030 MAIN ST, SUITE 1050, IRVINE, CA, 92614, US

(21) Appl. No.: 11/497,151

(22) Filed: Jul. 31, 2006

Related U.S. Application Data
(63) Continuation-in-part of application No. 10/866,153, filed on Jun. 14, 2004, now abandoned.

(30) Foreign Application Priority Data
Oct. 15, 2003 (TW) 092128526

(51) Int. Cl. A61B 1/00; A61B 1/04

(52) U.S. Cl. 600/101; 600/167; 600/109; 600/131; 600/102

(57) ABSTRACT

An endoscope apparatus includes a head portion for capturing images, a flexible connecting member made of optical fibers, and a body. The connecting member is used to transmit image data and control signals. A lens is provided in the head portion and adjustable to obtain sharp and clear images. The images are converted into image files and transmitted to an external data processor for data analysis. The optical fibers are flexible, such that the connecting member can be rolled and received in the body when it is not in use. A carrying member is provided on the body to allow a user to conveniently carry the endoscope apparatus. A disposable cover is used to cover the head portion and part of the connecting member that are to be in contact with an internal body cavity of a human, and a clamping member is provided to fix the cover in place.

This application is a continuation-in-part of application Ser. No. 10/886,153 filed Jun. 14, 2004.

### FIELD OF THE INVENTION

The present invention relates to medical devices for examining internal body cavities, and more particularly, to a portable endoscope apparatus with an adjustable focal length, which is capable of capturing and transmitting image data through optical fibers.

### BACKGROUND OF THE INVENTION

A conventional endoscope is made of a specialized and elongated tube, consisting of an imaging capturing device and a power supply. With the endoscope being connected to a screen, it allows internal body structures to be displayed on the screen and examined by the doctor for disease diagnosis when the endoscope is inserted into a body cavity. The organs having communication with the outside the body can be examined using the endoscope. For example, the endoscope is inserted through the mouth to the alimentary canal to examine the esophagus, stomach, and duodenum; and the intestines are examined by inserting the endoscope through the anus. If no inherent canal communicative with the outside is provided, surgery can be performed to form an entry for the endoscope. For examples, the laparoscope requires an opening being made on the abdominal cavity, and the skin around the joint should be cut open for accommodating the endoscope to examine the arthritis.

Basically, medical examination using the endoscope is slightly invasive. Insertion of endoscope into the body cavity may often discomfort a patient, even leading to shock in a severe case. Further, surfaces of the internal organs are delicate and might be wounded by the endoscope. Moreover, the conventional endoscope is an expensive apparatus, which needs to be cleaned and sterilized before the next use. However, since the endoscope is complex in structure, and the cleaning equipment for the endoscope is expensive, in case the standard cleaning procedure is not perfectly followed by an operator who is not familiar or careful with the cleaning equipment, the endoscope fails to be thoroughly and properly cleaned and sterilized, making patients who have ever been examined using this contaminated endoscope be in danger of cross-contamination. Therefore, how to develop an endoscope that would not be uncomfortable a patient, is easily operated and is free of cross-contamination is an important task to achieve.

In addition, remote areas such as rural areas are often short of medical supplies and can only rely on crude medical devices for medical examination and disease diagnosis, such that patients at these remote rural areas are usually not subject to good and timely treatments, making the patients' condition even worse. Although the government and private organizations periodically organize medical teams to provide more advanced medical devices and medicines to the remote areas, the effects achieved thereby on improvement in the medical condition of the remote areas are limited. A patient from the remote areas may require a detailed examination on his internal body at a large hospital having advanced and expensive examining devices such as an endoscope. However, these same devices are usually large in size and cannot be carried with the medical teams to the remote areas. Another important issue is the lack of medical human resources in the remote areas to give a correct diagnosis from image data taken by the endoscope even if it is brought to the remote areas. Furthermore, during transfer of the patients to the hospital, it is desirable to have a portable endoscope to timely examine the patients and transmit the image data captured by the endoscope to the hospital, which allows the doctors and nurses at the hospital to prepare necessary devices and treatments in advance to be used on the patients in response to the patients's condition from the transmitted image data, thereby increasing the chance of success in treating the patients and the working efficiency of the medical workers.

Therefore, the problem to be solved is to provide an endoscope, which is portable and capable of transmitting image data taken from a patient's internal canal through a network to a distant terminal where an accurate analysis or diagnosis can be performed.

### SUMMARY OF THE INVENTION

In order to solve the above drawbacks in the prior art, a primary objective of the present invention is to provide an endoscope apparatus, allowing the endoscope to be wound and received in a body of the apparatus, wherein the body is provided with a carrying member to allow a user to easily carry the endoscope apparatus that is thus more convenient to use.

Another objective of the invention is to provide an endoscope apparatus, which is formed with a head portion for capturing images, and a rotatable lens with a adjustable focal length in the head portion, to allow images to be accessed and clearly taken.

Still another objective of the invention is to provide an endoscope apparatus, which is formed with a biologically compatible and disposable cover for encapsulating a surface of the endoscope in contact with body cavities or canals of a patient, and a clamping member for holding the cover in position so as to avoid displacement of the cover when in contact with walls of the body cavities or canals and thereby protect the patient against cross-contamination.

A further objective of the invention is to provide an endoscope apparatus, which can transmit image data to an external data processor via wired or wireless transmission, allowing the external data processor to analyze the image data, and further transmit the image data through a network to a distant terminal for data integration.

In order to achieve the foregoing and other objectives, the present invention provides an endoscope apparatus, comprising: (1) a head portion comprising (a) an image capturing unit for capturing images, and (b) an illuminator module for providing light to the image capturing unit, wherein the image capturing unit comprises (i) a rotatable lens with an adjustable focal length, and (ii) a converting module for converting the images captured by the image capturing unit into digital image data; (2) a flexible connecting member connected to the head portion to form an endoscope, wherein the connecting member transmits power to the head portion for performing the image capturing, and also transmits the captured images from the head portion; and (3) a body for receiving the head portion and the connecting member therein, the body comprising (a) a carrying member for allowing a user to carry the endoscope apparatus via the carrying member, (b) a display unit for displaying the images captured by the image capturing unit, (c) a socket for accommodating a storage medium for storing data, (d) a power supply module for providing power to the endoscope apparatus, (e) an out port for being connected to an external data processor and performing data transmission, and (f) a control interface for adjusting an operating mode of the image capturing unit. The endoscope apparatus provided in the invention comprises an endoscope consisting of the head portion and the flexible connecting member to examine the condition of an internal body cavity of a human. The connecting member comprises a plurality of optical fibers and a cladding for covering the optical fibers, and is used as a transmission pathway for transmitting image data and control signals. The lens provided in the head portion can be adjusted to a proper focal length and rotated in position according to the user setting so as to obtain sharp images of the body cavity and even at blind angle positions. Moreover, since the optical fibers are flexible, the connecting member can be rolled up and received in the body. Furthermore, the carrying member provided on the body allows the user to conveniently carry the endoscope apparatus, making the endoscope apparatus portable and easy to use.

In addition, the endoscope apparatus further comprises a disposable cover and a clamping member, wherein the cover is used to cover part of the endoscope (including the head portion and part of the connecting member) that is to be inserted into the body cavity, and the clamping member is to fix the cover in place on the connecting member, such that cross-contamination between patients subject to the endoscope examination can be prevented. The images of the body cavity captured by the endoscope apparatus can be converted into image files and then transmitted to the external data processor where the image data are analyzed and integrated. Subsequently, the processed image files can be transmitted to a distant terminal such as medical center from the external data processor via a network connected to the data processor so as to perform a further analysis of the image files to give more thorough and accurate diagnosis.

### BRIEF DESCRIPTION OF THE DRAWINGS

The present invention can be more fully understood by reading the following detailed description of the preferred embodiments, with reference made to the accompanying drawings, wherein:

FIGS. 1(A) to 1(B) are schematic diagrams showing an endoscope apparatus in accordance with the present invention;

FIG. 2(A) is a schematic diagram showing a head portion and a flexible connecting member received in a body of the endoscope apparatus in accordance with the present invention;

FIG. 2(B) is a schematic diagram showing the head portion;

FIG. 2(C) is a block diagram showing the internal structure of an image capturing unit of the endoscope apparatus in accordance with the present invention;

FIG. 3 is a beveled view of the body of the endoscope apparatus in accordance with the present invention;

FIG. 4(A) is a schematic diagram showing a cover covering the head portion of the endoscope apparatus in accordance with the present invention;

FIG. 4(B) is a schematic diagram showing the endoscope apparatus in accordance with the present invention being used for examining a body cavity through the mouth; and

FIG. 5 is a block diagram showing the internal structure of the body of the endoscope apparatus in accordance with the present invention.

### DETAILED DESCRIPTION OF THE PREFERRED EMBODIMENT

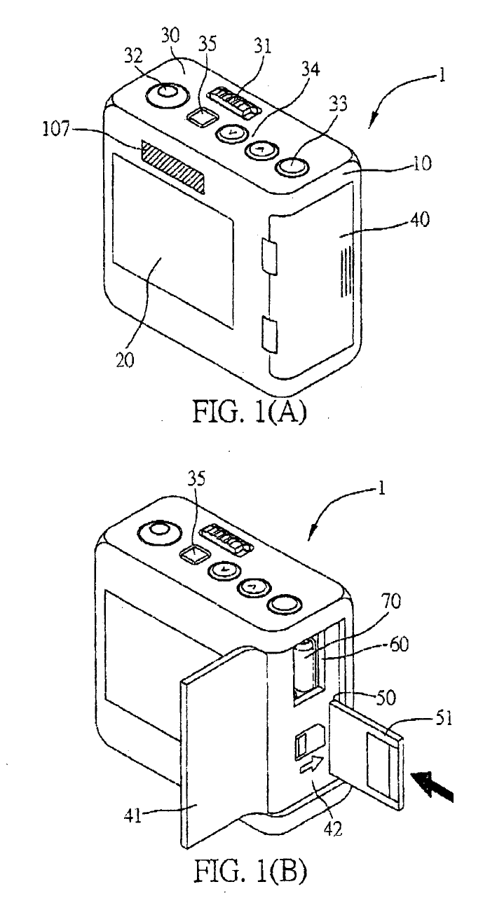

The preferred embodiment of an endoscope apparatus proposed in the present invention is described in detail with reference to FIGS. 1-5.

FIGS. 1(A) and 1(B) show the appearance of the endoscope apparatus 1 according to the present invention. As shown in FIG. 1(A), a body 10 of the endoscope apparatus 1 is provided with a display unit 20 such as liquid crystal display (LCD) screen for displaying images; a control interface 30 for providing operational functions; and a receiving unit 40 with a liftable cap, for receiving part of functioning components of the endoscope apparatus 1. On the control interface 30, there are provided a focal length controller 31 for adjusting a focal length of a lens (not shown) installed in a head portion 70 of the endoscope apparatus 1; an angle controller 32 for adjusting an angle of observation for the lens; a function switch button 33; a selector 34 and a retract button 35. The function switch button 33 allows the function items listed on the display unit 20 to be switched, and then the selector 34 allows the desired function to be selected by moving the cursor on the display unit 20 and also allows an operating mode of the selected function to be adjusted.

Moreover, as shown in FIG. 1 (B), the receiving unit 40 comprises the liftable cap 41 and a receiving space 42, wherein the receiving space 42 is formed with a socket 50 for accommodating a storage medium 51 (such as flash memory card) for storing image data, and an opening 60 via which the head portion 70 having image-capturing and image-transmitting functions can be pulled out. Also, a new unused cover (not shown) and a new unused clamping member (not shown) can be placed in the receiving space 42. The liftable cap 41 is used to close the receiving space 42 when the endoscope apparatus 1 is in use, so as to protect all the components received in the receiving space 42 against dust or other damages.

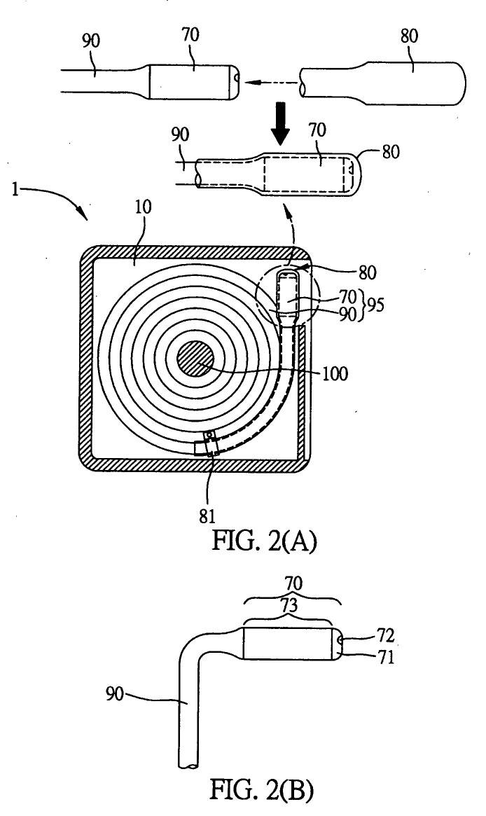

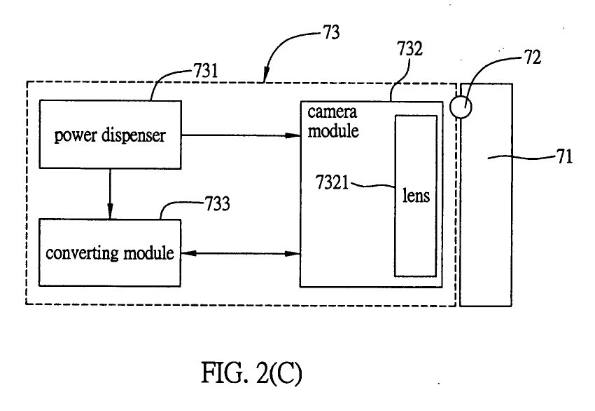

FIG. 2(A) shows the head portion 70 and a flexible connecting member 90 constituting an endoscope 95 that is to be inserted into a body cavity or canal for image capturing. When the use of the endoscope 95 is complete, the retract button 35 of FIG. 1(A) is pressed to retract or roll up the endoscope 95 back into the body 10. As shown in FIG. 2(A), the endoscope 95 when not in use is rolled with reference to an axis 100 as the center to be received in the body 10. When the endoscope 95 is ready to be inserted into a body cavity and/or canal, a disposable cover 80 having a length slightly longer than the body cavity and/or canal is covered on the head portion 70 and the connecting member 90, and a clamping member 81 is provided to fix the cover 80 in place on the connecting member 90. This is to avoid displacement of the cover 80 when in contact with a wall of the body cavity and/or canal and thereby prevent the occurrence of cross-contamination to a patient whose body cavity and/or canal is to be examined by the endoscope 95.

FIG. 2(B) shows the head portion 70 connected to the connecting member 90. The connecting member 90 is made of a plurality of optical fibers (made of SiO₂) and a cladding for covering and protecting the optical fibers, wherein the cladding is made of a material more rigid than the optical fibers and can be flexibly bent or shaped. For example, the cladding can be made of a plastic material such as polyvinyl chloride (PVC) or thermoplastic polyurethane (TPU). The head portion 70 comprises a transparent window 71 at the front end, an illuminator module 72 comprising at least one light emitting diode (LED), and an image capturing unit 73. The illuminator module 72 is to provide sufficient light for the image capturing unit 73 to take a sharp image from the body cavity and/or canal. As shown in FIG. 2(C), the image capturing unit 73 comprises a power dispenser 731, a camera module 732, and a converting module 733. The camera module 732 comprises a rotatable lens 7321 with an adjustable focal length. The power dispenser 731 is to dispense the supplied power to individual functioning components or modules in the image capturing unit 73. The camera module 732 allows the focal length of the lens 7321 to be adjusted and the lens 7321 to be rotated according to a command input from a user, so as to clarify an image captured through the transparent window 71 and pick up the clarified image that is then converted to digital image data by the converting module 733. The digital image data are subsequently transmitted via the flexible connecting member 90 to the display unit 20 of the body 10 where the image data can be displayed and observed by the user. During the image capturing process performed by the camera module 732, the illuminator module 72 located near the lens 7321 needs to provide sufficient light, such that the image captured by the camera module 732 would not be too dull to discriminate and is worth being analyzed as reference. The light source in this embodiment is not particularly limited, which can be a source of white light or infrared or a combination thereof. Typically the camera module 732 comprises at least one LED as the light source and is located around the lens 7321 to provide concentrative and sufficient light in the image capturing process.

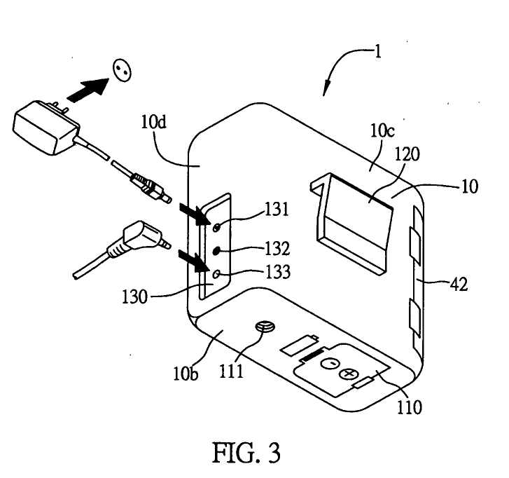

FIG. 3 is a beveled view of the body 10 of the endoscope apparatus 1. As shown, the bottom 10b of the body 10 is formed with a battery slot 110 for placing batteries (not shown) and a hole 111 for connecting a stand (not shown) to allow the endoscope apparatus 1 to be supported on the stand. One side 10c of the body 10 is provided with a carrying member 120 such as a clip that can be carried on the clothes of the user. The carrying member 120 closely abuts the side 10c with one end of the carrying member 120 connected to the body 10. The carrying member 120 is flexible such that the other end thereof not connected to the side 10c of the body 10 can be pulled apart from the body 10 to allow a belt or clothes of the user to be inserted between the carrying member 120 and the side 10c of the body 10, making the endoscope apparatus 1 easy to carry. In addition, another side 10d of the body 10 opposite to the side with the receiving space 42 is provided with an out port 130 that comprises a plug-in hole 131 for connecting the public supply of power or electricity, an antenna 132 for receiving/sending data during wireless transmission, and a cable socket 133 for connecting a transmission cable linked to an external data processor.

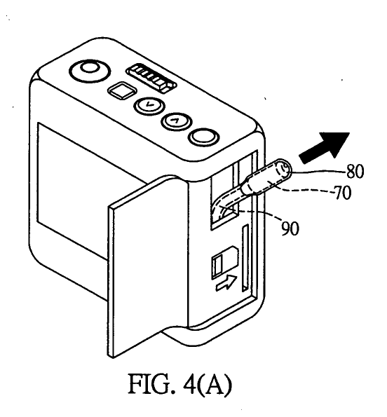

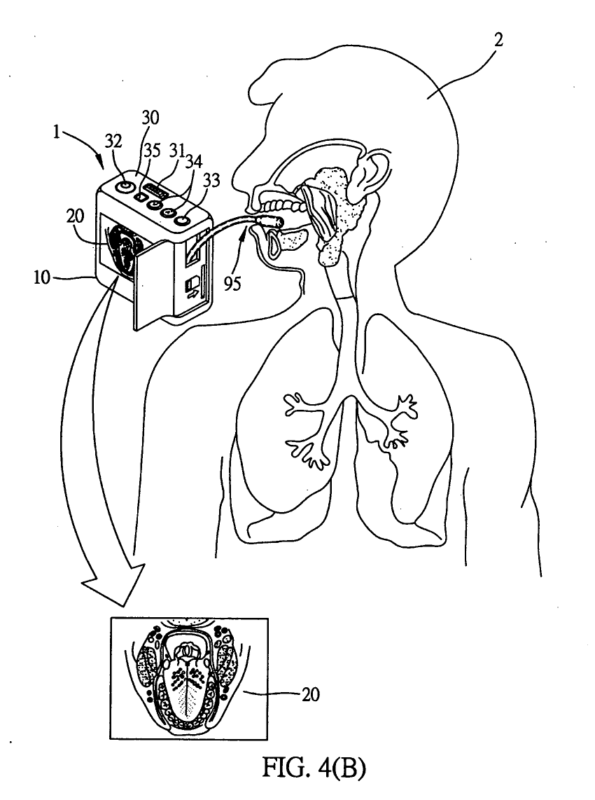

FIGS. 4(A) to 4(B) show the endoscope apparatus according to the present invention being used to examine a body cavity through the mouth. As shown in FIG. 4 (A), prior to the examination, firstly the head portion 70 and part of the flexible connecting member 90 are pulled out and covered by a cover 80 having a length slightly longer than the length of the body cavity to be examined. Subsequently, a clamping member 81 (FIG. 2(A)) is used to fix the cover 80 in place on the connecting member 90 such that the cover 80 can be prevented from displacement due to contact with the surface of the body cavity, thereby eliminating the occurrence of cross-contamination. After the cover 80 and the clamping member 81 are set up in place, as shown in FIG. 4(B), the endoscope 95 consisting of the head portion 70 and the connecting member 90 is gradually inserted through the mouth into the oral cavity of a human patient 2. During this process, the user can operate the focal length controller 31, angle controller 32 and function switch button 33 and selector 34 of the control interface 13 to adjust the sharpness of images in the oral cavity of the patient 2 that are captured by the camera module 732 and displayed on the display unit 20. For example, the user operates the focal length controller 31 to control the focal length of the lens 7321 of the camera module 732 to clarify the images of the oral cavity captured by the lens 7321. The user operates the angle controller 32 to rotate the lens 7321 to appropriate positions to take images of the oral cavity. The user operates the function switch button 33 to switch functions provided by the endoscope apparatus for example to switch to a function mode for adjusting light, and then uses the selector 34 to adjust (increase or reduce) the light emission strength from the illuminator module 72 around the lens 7321 so as to allow the illuminator module 72 to provide suitable and sufficient light for the camera module 732 to obtain clear images of the oral cavity. The image data obtained by the endoscope apparatus 1 can be transmitted in a wired or wireless manner to an external data processor (such as personal computer, notebook computer, personal digital assistant (PDA), etc.) to process the image data such as storage, analysis and printing. The external data processor can also be linked to a network to transmit the image data to a distant terminal where the data serve as reference for other associated expert people.

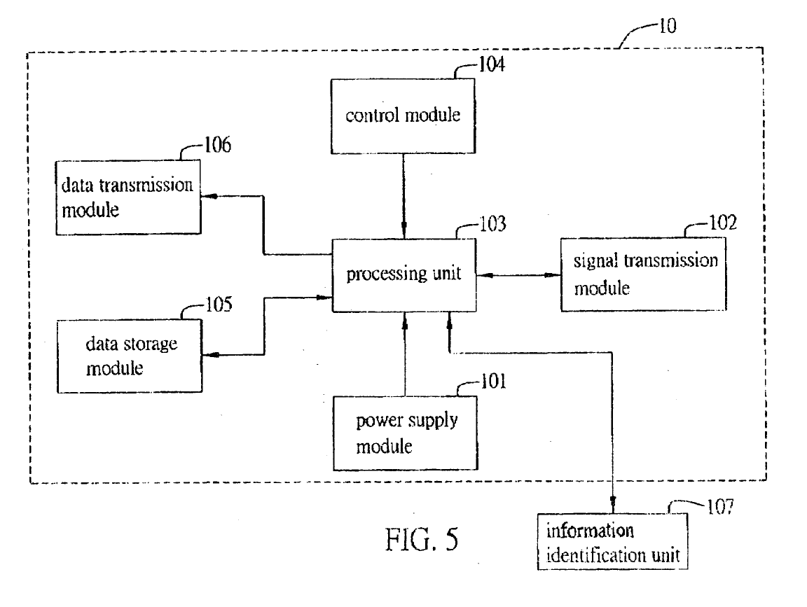

FIG. 5 shows the internal structure of the body 10 of the endoscope apparatus according to the present invention. As shown, the internal structure of the body 10 comprises a power supply module 101, a signal transmission module 102, a processing unit 103, a control module 104, a data storage module 105, and a data transmission module 106. The power supply module 101 supplies power to the other modules, and the processing unit 103 dispenses the appropriate power to each module. The power source can be batteries (not shown) provided in the endoscope apparatus, or the externally supplied public electricity, either of which is freely used.

The signal transmission module 102 receives image data from the image capturing unit 73 and sends out trigger signals for driving the functioning components in the image capturing unit 73 to operate. The control module 104 is electrically connected to the control interface 30 and thus allows the operating mode of the functioning components in the image capturing unit 73 to be adjusted according to the image data displayed on the display unit 20. Control signals output from the control module 104 are processed and digitized by the processing unit 103, and then are transmitted to the image capturing unit 73 via the signal transmission module 102 and the optical fibers in the flexible connecting member 90, so as to allow the image capturing unit 73 to capture images with desired position, clarity, shooting angle and lightness according to the user requirements, also to allow the image capturing unit 73 to transmit the captured images in the form of data to the signal transmission module 102. The signal transmission module 102 then transmits the received image data to the processing unit 103 where the image data are converted to image files according to the setting made by the user.

The image files are transmitted via the antenna 132 or the cable socket 133 to the external data processor through wireless or wired transmission. Alternatively, when the endoscope apparatus 1 is not connected to any external data processor, the processing unit 103 can store the image file in the data storage module 105 or storage medium 51, which allows the user to repeatedly execute the image files and display the captured images of the oral cavity on the display unit 20 for more detailed analysis.

By way of an additional improvement, the endoscope apparatus 1 may further comprise an information identification unit 107 for identifying or verifying the identity of the patient, or providing the patient's identity to an external device allowing the information of patient's identity received from the endoscope apparatus 1 to be analyzed or stored. To this end and as is best shown in FIG. 5, the information identification unit 107 is electrically connected to the processing unit 103, so that the endoscope apparatus 1 may be used to prevent anonymous, pseudonymous or unauthorized usage, and provide identity information of the patient. The information identification unit 107 can be applied to different kinds of identification such as fingerprint identification, iris scanning, face recognition, and voice (speaker) identification, and the location of the information identification unit 107 may be adapted according to the requirements of different kinds of identifications. For example, the information identification unit 107 may be a fingerprint identifier located on the body 10, so as to scan the patient's fingerprint or the user's fingerprint immediately, such that information of the user can be stored to the data storage module 105 or transmitted to the external device, wherein the information may be analyzed by the processing unit 103 or the external device. Moreover, the information identification unit 107 may also be used to identify or verify an internal identification component implanted in the body of the patient.

In another embodiment, the information identification unit 107 may be an ophthalmoscope module (not shown) for iris scanning identification, wherein the ophthalmoscope module can be provided on the signal transmission pathway between the processing unit 103 and the data storage module 105, and a plurality of ophthalmic image data, corresponding names and photos of patients can be stored in the data storage module 105. As a result, when a patient is receiving the endoscope examination, the doctor may firstly use the head portion 70 of the endoscope apparatus 1 to capture an ophthalmic image of the patient 2, allowing the captured ophthalmic image to be transmitted via the signal transmission module 102 to the processing unit 103 where the ophthalmic image is processed to produce an ophthalmic image data file. The ophthalmic image data file is input to the ophthalmoscope module, and then the ophthalmoscope module searches in the data storage module 105 for a record of stored ophthalmic image data matching the ophthalmic image data file. If the matched record of ophthalmic image data is found, the name and photo of the corresponding patient would be displayed on the display unit 20.

This allows the doctor to verify whether the patient 2 under the endoscope examination is the correct person corresponding to the personal details displayed on the display unit 20, so as to prevent the occurrence of misdiagnosis and ensure the rights of both the patient and the doctor. Moreover, the endoscope apparatus with the ophthalmoscope module can be also used as a portable identity recognition device by the Customs, the police force or other inspection organizations, which can improve the performance and efficiency of the identity verification process and thus help reduce the production of fake identifications for committing a crime.

In conclusion, the endoscope apparatus proposed in the present invention is characterized in comprising an endoscope consisting of a head portion and a flexible connecting member made by optical fibers, wherein the flexible connecting member can be rolled up and received in the body of the endoscope apparatus when it is not in use, such that the user can conveniently carry the endoscope apparatus. When the user intends to use the endoscope, the user needs to simply pull the endoscope out from the body and insert it to a body cavity such as mouth, nose, ear or rectum of a patient to perform examination. The head portion of the endoscope is provided with an illuminator module for providing sufficient light during the image capturing process so as to obtain sharp and clear images. The head portion further comprises a lens, allowing a focal length of the lens to be adjusted and the lens to be rotated according to the user requirements so as to obtain and display desirably clear images on a display unit to be observed by the user. Another characteristic feature of the endoscope apparatus is the use of a cover and a clamping member, wherein the cover is to cover part of the endoscope (including the head portion and part of the connecting member) that is to be in contact with the body cavity, and the clamping member is to fix the cover in place on the endoscope. The cover and clamping member are both disposable. Therefore, the occurrence of cross-contamination between patients subject to the endoscope examination can be avoided. A further characteristic feature of the endoscope apparatus is that the captured images of the body cavity can be transmitted through wireless or wire transmission to an external data processor for data processing and analysis to make the image data suitable as reference. In the case of wirelessly transmitting the image data, since the transmission distance is considered relatively short, the problem of generating a strong electromagnetic wave that interferes with surrounding medical devices would not occur.

Therefore, it is advantageous that the endoscope apparatus in the present invention with the endoscope being rolled and received in the body thereof is portable and convenient to carry by the user. Further, the images captured by the endoscope can be displayed to be observed and examined by the user and can be promptly transmitted to an external data processor for further analysis and processing. As a result, the endoscope apparatus can be used at any place and anytime, thereby increasing the convenience and working efficiency of carrying out the endoscope examination, and improving the time efficiency for performing any treatment required for the patient.

The present invention has been described using exemplary preferred embodiments. However, it is to be understood that the scope of the invention is not limited to the disclosed embodiments. On the contrary, it is intended to cover various modifications and similar arrangements. The scope of the claims, therefore, should be accorded the broadest interpretation so as to encompass all such modifications and similar arrangements.

1. An endoscope apparatus, comprising:
a head portion comprising an image capturing unit for capturing images, and an illuminator module for providing light to the image capturing unit, wherein the image capturing unit comprises:
a rotatable lens with an adjustable focal length; and
a converting module for converting the images captured by the image capturing unit into digital image data;
a flexible connecting member connected to the head portion to form an endoscope, wherein the connecting member transmits power to the head portion for performing image capturing, and also transmits the captured images from the head portion;
a disposable cover surrounding the head portion and at least some of the flexible connecting member to protect the patient against cross-contamination from the head portion and the at least some of the flexible connecting member;
a clamping member for fixing the disposable cover in place on the flexible connecting member to prevent a displacement of the cover relative to the head portion and the flexible connecting member;
a body for receiving the head portion and the flexible connecting member therein, the body comprising:
a receiving unit for receiving therewithin the disposable cover and clamping member prior to said cover surrounding said head portion and the at least some of said flexible connection member;
an information identification unit for identifying at least one of the user or the patient by providing identifying information that is personal to the user or the patient;
a display unit for displaying the images captured by the image capturing unit;
a signal transmission module for receiving data from the image capturing unit;
a socket for accommodating a storage medium for storing data;
a processing unit for processing data received from the signal transmission module and for being electrically connected to the information identification unit so as to allow the processing unit to process the data after the user's or the patient's identity has been identified by the information provided by the identification unit;
a power supply module for providing power to the endoscope apparatus;
an out port for transmitting data out of the endoscope apparatus; and
a control interface for adjusting an operating mode of the image capturing unit.

2. The endoscope apparatus of claim 1, which is for examining an internal body cavity of a human.

3. The endoscope apparatus of claim 2, wherein the body cavity is one selected from the group consisting of oral cavity, nasal cavity, ear cavity, and rectum cavity.

4. The endoscope apparatus of claim 1, wherein the connecting member comprises at least one optical fiber and a cladding for covering the optical fiber.

5. The endoscope apparatus of claim 6, wherein the cladding is made of a material more rigid than the optical fiber.

6. The endoscope apparatus of claim 1, wherein the head portion further comprises a transparent window.

7. The endoscope apparatus of claim 1, wherein the illuminator module is located near the lens.

8. The endoscope apparatus of claim 1, wherein the illuminator module comprises at least one light emitting diode (LED).

9. The endoscope apparatus of claim 1, wherein the display unit is a liquid crystal display (LCD).

10. The endoscope apparatus of claim 1, wherein the body of the endoscope apparatus is provided with a carrying member for allowing a user to carry the endoscope apparatus via the carrying member.

11. The endoscope apparatus of claim 1, wherein the storage medium is a flash memory card for storing the image data obtained by the image capturing unit.

12. The endoscope apparatus of claim 1, wherein the power supply module provides power from one of a public electricity supply and batteries to the endoscope apparatus.

13. The endoscope apparatus of claim 1, wherein the out port transmits the data to an external data processor via wired or wireless transmission, and the data processor is one selected from the group consisting of personal computer, notebook computer, and personal digital assistant (PDA).

14. The endoscope apparatus of claim 16, wherein the out port is provided with at least one cable socket and an antenna to transmit the data via the wired or wireless transmission.

15. The endoscope apparatus of claim 1, wherein the control interface allows at least one of the focal length of the lens to be adjusted, the lens to be rotated, and light strength from the illuminator module to be controlled.

16. The endoscope apparatus of claim 1, wherein an ophthalmoscope module for identity recognition is provided in the body of the endoscope apparatus.

17. The endoscope apparatus of claim 1, wherein the flexible connecting member is adapted to be rolled up so that the head portion and the connecting member can be received in the body.
